(19) United States
(12) Patent Application Publication
(10) Pub. No.: US 2024/0065756 A1
(43) Pub. Date: Feb. 29, 2024

(54) PULSE ABLATION CATHETER FOR TREATING PULMONARY ARTERIAL HYPERTENSION

(71) Applicant: Yan'an Hospital of Kunming City, Kunming (CN)

(72) Inventors: Hailong DAI, Kunming (CN); Zhicheng JING, Beijing (CN); Zhiyan HAN, Beijing (CN); Xuefeng GUANG, Kunming (CN); Xu ZHOU, Beijing (CN)

(21) Appl. No.: 18/499,597

(22) Filed: Nov. 1, 2023

Related U.S. Application Data
(63) Continuation of application No. PCT/CN2022/108800, filed on Jul. 29, 2022.

(30) Foreign Application Priority Data
Aug. 11, 2021 (CN) 202110919334.9

(51) Int. Cl. A61B 18/14

(52) U.S. Cl. CPC A61B 18/1492; A61B 2018/00577

(57) ABSTRACT

A pulse ablation catheter for treating pulmonary arterial hypertension includes an ablation catheter part and an energy platform, where the ablation catheter part includes a movable outer sheath tube, an ablation catheter, a functional guide core, a pulse ablation part and an operating part; the operating part controls the pulse ablation part to slide axially on the functional guide core in a center of the ablation catheter, and controls strip-shaped arches in a middle of the pulse ablation part to arch or fold in a circumferential direction so as to perform pulse ablation through pulse electrode sheets and the energy platform. The pulse ablation catheter for treating pulmonary arterial hypertension of the application performs pulse ablation through the pulse ablation catheter with controllable frequency and ablation end shape to treat pulmonary arterial hypertension.

### CROSS-REFERENCE TO RELATED APPLICATIONS

This application is a continuation of PCT/CN2022/108800, filed on Jul. 29, 2022 and claims priority to Chinese Patent Application No. 202110919334.9, filed on Aug. 11, 2021, the entire contents of which are incorporated herein by reference.

### TECHNICAL FIELD

The application relates to the technical field of medical instruments, and in particular to a pulse ablation catheter for treating pulmonary arterial hypertension.

### BACKGROUND

Pulmonary arterial hypertension is a kind of vascular disease mainly involving pulmonary arterioles. The pulmonary artery vascular remodeling leads to hemodynamic changes of cardiopulmonary circulation, pulmonary arterial hypertension, right heart hypertrophy and functional failure. The gold standard for the diagnosis of pulmonary arterial hypertension is that the average pulmonary arterial pressure measured by the right cardiac catheter at sea level and at rest is ≥25 mmHg, while the wedge pressure of pulmonary arterioles is ≤15 mmHg and the pulmonary vascular resistance is ≥3 Wood units. Some experimental data show that pulmonary arterial hypertension is related to the increased excitability of peripheral sympathetic nerve around the pulmonary artery and abnormal activity of pulmonary baroreceptor. Blocking peripheral sympathetic nerve around the pulmonary artery or permanently destroying the structure and function of baroreceptor is able to reduce the pulmonary artery pressure, which will become a breakthrough technology for treating pulmonary arterial hypertension.

Pulsed electric field ablation refers to the action of high-voltage electric pulses on phospholipid bilayer of cell membrane in a short time, which leads to the formation of transmembrane potential, resulting in unstable potential, irreversible penetrating damage to cell membrane, and nano-scale pores, which leads to the change of cell membrane permeability, destruction of intracellular homeostasis, and ultimately leads to apoptosis.

Pulsed electric field ablation has the following characteristics: firstly, pulsed electric field ablation is able to preserve extracellular matrix. Ablation technology based on heat conduction relies on coagulation necrosis and extends coagulation necrosis to the lethal temperature of cells. Although cryoablation is able to avoid direct ablation of extracellular matrix, it has no selectivity for cell destruction and is able to affect the structure of target blood vessels. However, pulsed electric field ablation is able to maintain the integrity of tissue matrix in its ablation area and avoid damage to adjacent tissues such as coronary artery and phrenic nerve. Secondly, ablation threshold is tissue-specific, so it is able to specifically ablate some specific tissues (such as myocardium and pulmonary artery). The ablation threshold of myocardial tissue is lower than that of many other tissues, which is able to avoid damage to adjacent tissues (esophagus or phrenic nerve, etc.) while ablating myocardial cells. Thirdly, compared with traditional radiofrequency ablation, pulsed electric field ablation is able to cause extensive myocardial injury without relying on catheter contact force. Lastly, pulsed electric field ablation speed is extremely fast, often in milliseconds or even less. To sum up, pulsed electric field ablation may be safer and more effective, and greatly shorten the operation time.

At present, the treatment of pulmonary arterial hypertension still adopts the radiofrequency ablation or cryoablation, and there is no pulse ablation catheter for pulmonary arterial hypertension, which makes the treatment of pulmonary arterial hypertension still limited.

### SUMMARY

In view of the above problems existing in the prior art, the application provides a pulse ablation catheter for treating pulmonary arterial hypertension, and pulse ablation is performed through the pulse ablation catheter with controllable frequency and ablation end shape to treat pulmonary arterial hypertension.

In order to achieve the above technical objectives and achieve the above technical effects, the application is realized through the following technical scheme.

A pulse ablation catheter for treating pulmonary arterial hypertension includes an ablation catheter part and an energy platform, where the ablation catheter part includes a movable outer sheath tube, an ablation catheter, a functional guide core, a pulse ablation part and an operating part. The movable outer sheath tube moves axially outside the ablation catheter, and the position and shape of the pulse ablation part of the ablation catheter part are controlled by the operating part at the rear end of the ablation catheter part. The pulse ablation part fits the ablation catheter or arches to form a hollow spherical shape, and the pulse ablation part is connected with the energy platform for pulse ablation.

Optionally, the ablation catheter is movably connected with the movable outer sheath tube, the rear end of the movable outer sheath tube is the operating part. A functional guide core is arranged in the ablation catheter. The front end of the pulse ablation part is a movable end. The pulse ablation part is controlled by the operating part to slide axially on the functional guide core in the center of the ablation catheter, and the middle of the pulse ablation part is provided with a plurality of strip-shaped arches. The strip-shaped arches are able to arch in a circumferential direction.

Optionally, the operating part at a rear end of the pulse ablation part is fixed with a functional guide core.

Optionally, each of the strip-shaped arches is provided with pulse electrode sheets, the pulse electrode sheets wrap corresponding one of the strip-shaped arches at intervals. The pulse electrode sheets are powered by the energy platform connected with the rear end of the functional guide core, and the tail end of the pulse ablation part is integrated with the ablation catheter.

Optionally, the energy platform is connected with the ablation catheter, and the energy platform includes a host, a display end and an adjusting end

Optionally, the functional guide core is a hollow inner core for the internal guide wire to pass through, and is also used for monitoring pulmonary arterial pressure.

The application has following beneficial effects.

According to the application, the pulse ablation catheter for treating pulmonary arterial hypertension includes the ablation catheter part and the energy platform, where the ablation catheter part includes the movable outer sheath tube, the ablation catheter, the functional guide core, the pulse ablation part and the operating part. Through the operating part, the pulse ablation part is controlled to slide axially on the functional guide core in the center of the ablation catheter and at the same time, the strip-shaped arches in the middle of the pulse ablation part are controlled to arch or fold in a circumferential direction, so that pulse ablation is performed through pulse electrode sheets and the energy platform.

The pulse ablation catheter for treating pulmonary arterial hypertension of the application performs pulse ablation through the pulse ablation catheter with controllable frequency and ablation end shape to treat pulmonary arterial hypertension.

Of course, it is not necessary for any product of the present application to have all the advantages mentioned above at the same time when applied.

### BRIEF DESCRIPTION OF THE DRAWINGS

In order to explain the technical scheme of the embodiment of the present application more clearly, the drawings needed for the embodiment description will be briefly introduced below. Obviously, the drawings in the following description are only some embodiments of the present application. For ordinary technicians in the field, other drawings may be obtained according to these drawings without creative work.

FIG. 1 is a schematic structural diagram of a pulse ablation catheter for treating pulmonary arterial hypertension according to an embodiment of the present application.

FIG. 2 is a schematic structural diagram of an ablation catheter part according to an embodiment of the present application.

### DETAILED DESCRIPTION OF THE EMBODIMENTS

In the following, the technical scheme in the embodiment of the application will be clearly and completely described with reference to the drawings. Obviously, the described embodiments are only a part of the embodiments of the application, but not the whole embodiments. Based on the embodiments in the present application, all other embodiments obtained by ordinary technicians in the field without creative work belong to the scope of protection of the present application.

### Embodiment 1

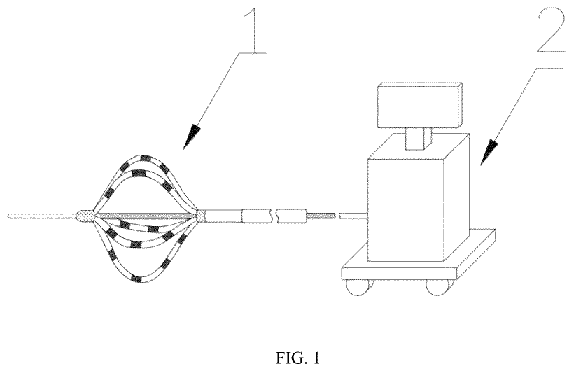

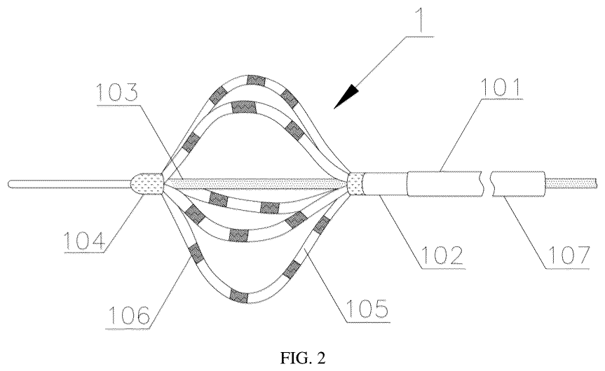

As shown in FIG. 1 and FIG. 2, the pulse ablation catheter for treating pulmonary arterial hypertension includes the ablation catheter part 1 and the energy platform 2. The ablation catheter part 1 includes the movable outer sheath tube 101, the ablation catheter 102, the functional guide core 103, the pulse ablation part and the operating part 107. The movable outer sheath tube 101 moves axially outside the ablation catheter 102, and the position and shape of the pulse ablation part of the ablation catheter part 1 are controlled by the operating part 107 at the rear end of the pulse ablation part 1. The pulse ablation part fits the ablation catheter 102 or arches to form a hollow spherical shape, and the pulse ablation part is connected with the energy platform 2 for pulse ablation.

The ablation catheter 102 is movably connected with the movable outer sheath tube 101. The rear end of the movable outer sheath tube 101 is provided with the operating part 107. The functional guide core 103 is arranged in the ablation catheter 102. The front end of the pulse ablation part is the movable end 104. The pulse ablation part is controlled by the operating part 107 to slide axially on the functional guide core 103 in the center of the ablation catheter. The middle of the pulse ablation part is provided with a plurality of strip-shaped arches 105. The strip-shaped arches 105 are able to be arched in the circumferential direction.

The operating part 107 at a rear end of the pulse ablation part is fixed with a functional guide core.

Each of the strip-shaped arches 105 is provided with pulse electrode sheets 106, the pulse electrode sheets 106 wrap corresponding one of the strip-shaped arches at intervals. The pulse electrode sheets 106 are powered by the energy platform 2 connected with the rear end of the functional guide core 103, and the tail end of the pulse ablation part is integrated with the ablation catheter 102.

The energy platform 2 is connected with the ablation catheter 102, and the energy platform includes the host, the display end and the adjusting end.

The functional guide core 103 is a hollow inner core for the internal guide wire to pass through.

### Embodiment 2

An operation method of pulse ablation catheter for treating pulmonary arterial hypertension is provided.

The operation method applies the pulse ablation catheter for treating pulmonary arterial hypertension described in the embodiment 1. The rear end of the ablation catheter is connected with the energy platform. Through the guide of the guide wire, the functional guide core, the ablation catheter and the movable outer sheath tube are transported to the corresponding positions. After the ablation catheter reaches the corresponding positions, the movable outer sheath tube moves backward to expose the pulse ablation part of the ablation catheter part, and the functional guide core is controlled by the operating end to be pulled backward. Because the front end of the functional guide core is fixed with the pulse ablation part, and the rear end of the pulse ablation part is integrated with the ablation catheter, the strip-shaped arches of the pulse ablation part are arched to form the hollow spherical shape. And then the functional guide core is fixed to keep the strip-shaped arches be the hollow spherical shape, and the pulse ablation part reaches designated position for pulse ablation.

After the ablation is completed, the pulse ablation part is retracted through the operating end, and the strip-shaped arches arching to be the hollow spherical shape are folded through the functional guide core, so that the pulse ablation part fits the ablation catheter and exits.

### Embodiment 3

The pulse ablation catheter used for treating pulmonary arterial hypertension of the present application includes the ablation catheter and the energy platform. The ablation catheter includes the movable outer sheath tube, the ablation catheter, the functional guide core, the pulse ablation part, and the operating part. The operating part controls the pulse ablation part to slide axially on the functional guide core in the center of the ablation catheter while controlling the strip-shaped arches in the middle of the pulse ablation part to arch or fold in a circumferential direction and then pulse ablation is performed through pulse electrode sheets and the energy platform.

The pulse ablation catheter used in the treatment of pulmonary arterial hypertension of the present application carries out pulse ablation through the pulse ablation catheter with controllable frequency and ablation end shape to treat pulmonary arterial hypertension.

The preferred embodiments of the present application disclosed above are only used to help illustrate the present application. The preferred embodiments do not describe all the details in detail, and they are not all embodiments of the application. Obviously, many modifications and changes may be made according to the contents of this specification. These embodiments are selected and described in detail in this specification in order to better explain the principle and practical application of the present application, so that those skilled in the technical field are able to better understand and utilize the present application. The application is limited only by the claims and their full scope and equivalents.

What is claimed is:

1. A pulse ablation catheter for treating pulmonary arterial hypertension, comprising an ablation catheter part and an energy platform, wherein the ablation catheter part comprises a movable outer sheath tube, an ablation catheter, a functional guide core, a pulse ablation part and an operating part, the movable outer sheath tube moves axially outside the ablation catheter, and the operating part at a rear end of the ablation catheter part is capable of controlling a position and a shape of the pulse ablation part of the ablation catheter part, the pulse ablation part fits the ablation catheter or arches to form a hollow spherical shape, and the pulse ablation part is connected with the energy platform for pulse ablation.

2. The pulse ablation catheter for treating the pulmonary arterial hypertension according to claim 1, wherein the ablation catheter is movably connected with the movable outer sheath tube, a rear end of the movable outer sheath tube is the operating part, the functional guide core is arranged in the ablation catheter, a front end of the pulse ablation part is a movable end, and the operating part is capable of controlling the pulse ablation part to slide axially along the functional guide core in a center of the ablation catheter, and a middle of the pulse ablation part is provided with a plurality of strip-shaped arches, and the strip-shaped arches are capable of arching in a circumferential direction.

3. The pulse ablation catheter for treating the pulmonary arterial hypertension according to claim 2, wherein the operating part at a rear end of the pulse ablation part is fixed with a functional guide core.

4. The pulse ablation catheter for treating the pulmonary arterial hypertension according to claim 2, wherein each of the strip-shaped arches is provided with pulse electrode sheets, the pulse electrode sheets wrap corresponding one of the strip-shaped arches at intervals, the pulse electrode sheets are powered by the energy platform connected with a rear end of the functional guide core, and a tail end of the pulse ablation part is integrated with the ablation catheter.

5. The pulse ablation catheter for treating the pulmonary arterial hypertension according to claim 1, wherein the energy platform is connected with the ablation catheter, and the energy platform comprises a host, a display end and an adjusting end.

6. The pulse ablation catheter for treating the pulmonary arterial hypertension according to claim 1, wherein the functional guide core is a hollow inner core for an internal guide wire to pass through, and is also used for monitoring pulmonary arterial pressure.
